(19) United States
(12) Patent Application Publication
(10) Pub. No.: US 2007/0185583 A1
(43) Pub. Date: Aug. 9, 2007

(54) Joint prosthesis, use of such and a method for the application of a joint prosthesis

(76) Inventor: Per-Ingvar Branemark, Molndal (SE)

Correspondence Address: GREENBERG TRAURIG, LLP, 1750 TYSONS BOULEVARD, 12TH FLOOR, MCLEAN, VA, 22102, US

(21) Appl. No.: 11/569,908

(22) PCT Filed: May 28, 2005
(86) PCT No.: PCT/SE05/00807

§ 371 Date: Dec. 1, 2006

(30) Foreign Application Priority Data
Jun. 1, 2004 (SE) 0401400-7

(51) Int. Cl. A61F 2/30

(52) U.S. Cl. 623/21.15; 623/23.41

(57) ABSTRACT

The invention relates to a joint prosthesis comprising a first anchoring element (1a), a second anchoring element (1b) and a joint body (9) of an elastic material. The joint body (9) is attached to the two anchoring elements (1a1b). These are arranged to be secured into a respective bone having a respective anchoring axis (A, B). According to the invention, the anchoring axes (1a, 1b) are arranged so that they do not coincide with each other when the joint prosthesis is in an unloaded state. The invention also relates to a use of the invented joint prosthesis as well as a method for the application of a joint prosthesis.

### FIELD OF THE INVENTION

The present invention relates in a first aspect to a joint prosthesis comprising a first anchoring element, a second anchoring element and a joint body of an elastic material, which joint body is arranged so that the joint prosthesis is deflectable at least in a first plane of bending and which joint body is attached to said first and second anchoring elements, each of which anchoring elements are arranged to be secured into a respective bone having a respective anchoring axis running in the longitudinal axis of the respective bone, which anchoring axis do not coincide with another when the joint prosthesis is in an unloaded state.

In a second aspect, the invention relates to a use of the invented joint prosthesis.

In a third aspect, the invention relates to a method for the application of a joint prosthesis of a type comprising an elastic joint body attached to a first and a second anchoring element, the anchoring elements being secured into a respective bone having a respective anchoring axis running in the longitudinal axis of the respective bone.

### BACKGROUND OF THE INVENTION

A joint prosthesis of the mentioned type is previously known by, for instance, U.S. Pat. No. 5,011,497.

The known joint prosthesis comprises a joint body having a web of an elastomeric material, arranged between a pair of pins, which are inserted in one each of a pair of tubular screws for securing in the bones between which the joint prosthesis is to be arranged, the pins being secured in the web by means of flanges embedded in the elastomeric material. Pins and screws consist of a biocompatible material.

The known joint prosthesis is expedient in many respects and provides a good functional reliability with a simple construction. However, it has certain limitations that occasionally may entail disadvantages.

Thus, the joint prosthesis according to U.S. Pat. No. 5,011,497 is constructed so that the pins being secured into the respective bone are located in alignment with each other when the prosthesis is unloaded. This entails that the elastic forces of the joint body aims to keep the joint straight.

However, for certain patients there is a need for the joint to be slightly angled in the unloaded state. Such is the case, for instance, for patients with rheumatoid arthritis where there is an inherent tendency that the joint, for instance a finger joint, becomes angled. In a conventional joint prosthesis, for instance of the type previously known by U.S. Pat. No. 5,011,497, the joint is at every instant, subjected to a force that counteracts the angulation.

Through EP1 300 122 and U.S. Pat. No. 6,319,284 it is further known joint prostheses where the anchoring axes are angled relative each other.

The known joint prostheses are arranged to allow angling only in one plane of bending. In many cases there is a need also to obtain a controlled angling also laterally, i.e. perpendicular to the main moving direction of the joint.

The object of the present invention is to meet said need.

### SUMMARY OF THE INVENTION

In a first aspect of the invention, the object set up has been attained by the fact that a joint prosthesis of the kind defined in the preamble of claim 1 has the special features that the joint body is arranged so that the joint prosthesis is deflectable also in a second plane of bending, perpendicular to said first plane of bending, the moment of resistance of the joint body for bending in the first plane of bending being smaller than the moment of resistance thereof in the second plane of bending.

Thanks to these axes do not coinciding with each other, i.e., being either parallel-displaced or angled, the elasticity of the joint body will entail that the joint prosthesis, when installed in a patient, aims to give the joint an angled position, which can be adapted to the angulation that feels convenient for the patient. A big advantage of a pre-bent joint is that the tensile stresses upon bending can be lowered by the fact that the pre-bent joint does not need to be bent as much as the straight joint. This becomes particularly evident in simple everyday manipulations where the angle of the MCP joint usually is considerably smaller than the ROM thereof. Since the joint body is so arranged that the joint prosthesis is deflectable also in another plane of bending, perpendicular to the first plane of bending, the joint becomes controllable in space in a way not allowed by conventional joint prostheses. In this way, the need for, to a certain extent, also obtaining a controlled angulation force laterally, i.e., transverse to the actual direction of motion of the joint, is met. Since the moment of resistance of the joint body for bending in the first plane of bending is smaller than the moment of resistance thereof in the second plane of bending, the joint prosthesis gets properties being optimally adapted to allow large bending in the normal bending direction of the joint, where normally a large bending angle should be attainable, while the bending capability in the lateral direction becomes more limited and adapted to the limited bending need being at hand laterally.

According to a preferred embodiment, the anchoring axes form an angle to each other in a projection in the first plane of bending. In most cases, this constitutes the most expedient alternative for bringing about that the joint, when installed, aims to assume an angled position.

According to an additional preferred embodiment, the angle is in the interval of 5-30 °, preferably in the interval of 10-20°. These are the angular intervals most often being appropriate for the inclination of the joint, and therefore it is expedient to have the angle of the axes to each other within this interval.

According to an additional preferred embodiment, the anchoring axes form an angle to each other also in the second plane of bending, the first angle being larger than the second angle. In this way, an angulation capability is readily obtained also in the second plane, and which by the mentioned adaption of the angles to each other entails that the angle of inclination in the second plane of bending becomes more limited and adapted to the bending need in that direction.

According to an additional preferred embodiment, the two anchoring axes are parallel to each other. This is an alternative way to ensure that the joint prosthesis, when being installed, is given a prestress that aims to put the joint at an angle.

According to an additional preferred embodiment, the joint body comprises a first bordering surface and a second bordering surface located on one side each of the projection of the anchoring axes in the first plane of bending, which bordering surfaces in a cross section in said first plane of bending have different outlines and each outline having at least one part being concave up against said projection of the bending axes.

With such an asymmetrical profile of the bordering surfaces of the joint body, a predetermined function between degree of deflection and bending force is attained, and which becomes different upon bending in one or the other direction from the neutral position. Hence, the joint prosthesis according to this embodiment will meet the normally occurring need in that it should be easier to bend the joint in one direction than in the other.

According to an additional preferred embodiment, the two bordering surfaces and the respective concave parts thereof have different radii of curvature. This is a simple and expedient way to provide the differing bending characteristics desired for the bending in one or the other direction.

According to an additional preferred embodiment, the distances from the anchoring axis of the first anchoring element are different to the respective concave part of the two bordering surfaces. This is an alternative simple way to achieve the desired asymmetry. It may also advantageously constitute a complement to the arrangement above having different radii of curvature of the concave parts.

According to an additional preferred embodiment, the concave parts are located closer to the first anchoring element than the second anchoring element. Thereby, asymmetry is introduced also in the longitudinal direction of the joint so that the asymmetrical bending characteristic of the joint prosthesis is further affected to the desired extent.

According to an additional preferred embodiment, at least one of the anchoring elements comprises an end flange secured in the joint body, which flange has a curved shape including a convex surface facing the central part of the joint body. Suitably, both anchoring elements have this design. An end flange at the anchoring element and embedded in the joint body is also present at the joint body according to U.S. Pat. No. 5,011,497 mentioned by way of introduction, although there, the same has a plane shape. The convex curvature entails a more favourable holding effect between the end flange and the joint body in which it is embedded, particularly when the joint prosthesis is adapted to assume a certain angulation in rest.

According to an additional advantageous embodiment, the joint prosthesis is a finger joint prosthesis. It is for finger joints the need forming the basis of the invention is most pronounced, and where the advantages that the invention involves are most valuable.

The above-mentioned preferred embodiments of the invented joint prosthesis are defined in the claims depending on claim 1.

In the second aspect of the invention, the stated object is attained by the fact that the invented joint prosthesis is used to replace a defect joint of a person.

In the third aspect of the invention, the stated object is attained by the fact that a method of the type defined in the preamble of claim 13 comprises the special measure of arranging the joint body so that the joint prosthesis is deflectable also in a second plane of bending, perpendicular to said first plane, the moment of resistance of the joint body for bending in the first plane of bending being smaller than the moment of resistance thereof in the second plane of bending.

Preferred embodiments of the invented method are defined in the claim depending on claim 13.

With the invented method and the preferred embodiments thereof, advantages are gained of a type corresponding to what have been described above for the invented joint prosthesis and preferred embodiments of the same.

The invention is more closely explained by the subsequent detailed description of advantageous embodiment examples of the invented joint prosthesis, reference being made to the appended drawing Figures.

### BRIEF DESCRIPTION OF THE DRAWINGS

FIG. 1 is a longitudinal section through a previously known joint prosthesis.

FIG. 2 is a longitudinal section through an example of a joint prosthesis according to the invention.

FIG. 3 is a longitudinal section through an additional example of a joint prosthesis according to the invention.

FIG. 4 is a longitudinal section through yet an example of a joint prosthesis according to the invention, which section is perpendicular to the one in FIGS. 1-3.

FIGS. 5-9 are longitudinal sections through additional examples of joint prostheses according to the invention.

FIG. 10 is a side view of an example of a joint prosthesis according to the invention including indications of dimension.

FIG. 11 is an end view of the joint prosthesis in FIG. 10.

FIG. 12 is a side view of a detail of an example of a joint prosthesis according to the invention.

FIG. 13 is an end view of the detail in FIG. 12.

### DETAILED DESCRIPTION OF THE DRAWINGS

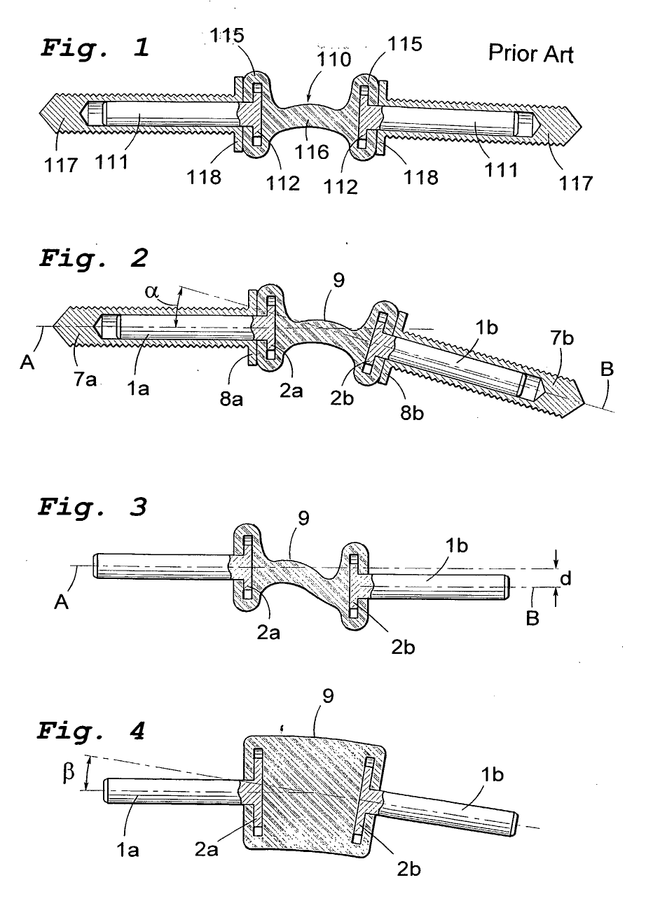

FIG. 1 is a vertical cross-section through a previously known joint prosthesis in this case a finger joint prosthesis. The joint prosthesis comprises a joint body 110 of an elastomer, which may consist of silicone rubber or polyurethane. This joint body is connected to a pair of anchoring elements in the form of pins 111 of a biocompatible material, preferably titanium, which are secured in the joint body by the fact that the same is injection moulded around end flanges 112 of the pins, so that these end flanges are completely embedded in the elastomeric material.

Each pin 111 is inserted in a tubular screw 117, which likewise should be composed of a biocompatible material and suitably is of the same nature as the pins, i.e., it should be composed of titanium. The screw has an end flange 118 and is screwed into the channel fixedly into the bone, which entails that the bone is not decomposed because of uneven load and that the prosthesis can be subjected to larger loads for longer periods, so that also younger patients living an active life can get a prosthesis that works considerably better for a long period than prostheses of known embodiment. The boring in the screw is sufficiently long for the pin not to touch the bottom of the same when it is fully inserted into the boring. The assembly of the joint prosthesis is facilitated by the possibility of inserting the pins 111 into the screws 117, when the same have been secured into the bones. After the assembly, the pins are kept pressed in the screws by the elastic joint body 110 arranged between them. Possibly, the pins may be locked in the screws after the assembly. Since no movement takes place between the pins and the screws during the use of the prosthesis, the wear of the prosthesis becomes minimal, whereby the service life will be large and the risk of infections and irritations caused by particles from the prosthesis entering into the body tissues is totally eliminated in practice. Another advantage is that, should the joint prosthesis for some reason not work satisfactory, the joint body with appurtenant pins can be exchanged easily by a simple operation with the screws still being secured in the bones. The pin may be cylindrical, so that it is rotatable in the screw, whereby the prosthesis becomes easier to adjust, but the rotatability also entails the risk of the prosthesis becoming angularly displaced out of the proper position thereof during use. Even if the rotatable embodiment becomes less costly than an embodiment where means are arranged to prevent rotation, it may therefore possibly be preferable that the pin is guided in the boring in such a way that it cannot be rotated therein. This can be attained by the fact that a projection on the pin or in the boring is displaceably received in a groove in the boring or the pin, respectively, or by the fact that the pin and the boring have a mating out-of-round shape. For instance, the pin and the boring may be of a hexagonal cross-section shape, which entails the advantage that upon tightening, the screw can be tightened by means of a pin spanner engaging in the boring thereof. In other cases, the screw may be formed with a screwdriver slot or with a hexagonal flange, in order to allow the engagement of the same upon tightening. Thanks to the elastic joint body 10, the joint prosthesis is non-locking.

FIG. 2 shows a finger joint prosthesis according to an embodiment example of the invention. The joint prosthesis showed in FIG. 2 has fundamental similarities with the known joint prosthesis according to FIG. 1. Thus, the joint prosthesis is provided with anchoring elements 1a, 1b arranged to be insertable into tubular screws 7a, 7b. The difference between the joint prosthesis according to the invention showed in FIG. 2 and the previously known joint prosthesis showed in FIG. 1 is that the anchoring axes A, B of the anchoring elements 1a, 1b in the joint prosthesis according to the invention form an angle a to each other when the joint prosthesis is unloaded. In the example shown, the angle α=15°. The elasticity of the joint body 9 aims to direct the joint prosthesis into this neutral position.

FIG. 3 shows an alternative embodiment example of a finger joint prosthesis according to the invention. In this example, the two anchoring axes A, B are parallel but displaced a distance d in relation to each other in the unloaded state. When a prosthesis according to this example is installed with the anchoring elements, i.e., the pins 1a, 1b, in the respective channel into two bones located opposite to each other, the displacement d between the axes A, B will cause a forced angulation of the joint.

In FIG. 4, an example of a finger joint prosthesis is shown in a longitudinal section perpendicular to the cross-sections shown in FIGS. 1-3, with the anchoring axes A, B forming an angle β to each other in the unloaded state also in a second plane of bending. The inclination in this plane causes a forced inclination in the lateral direction. In the example shown the angle p β=7°.

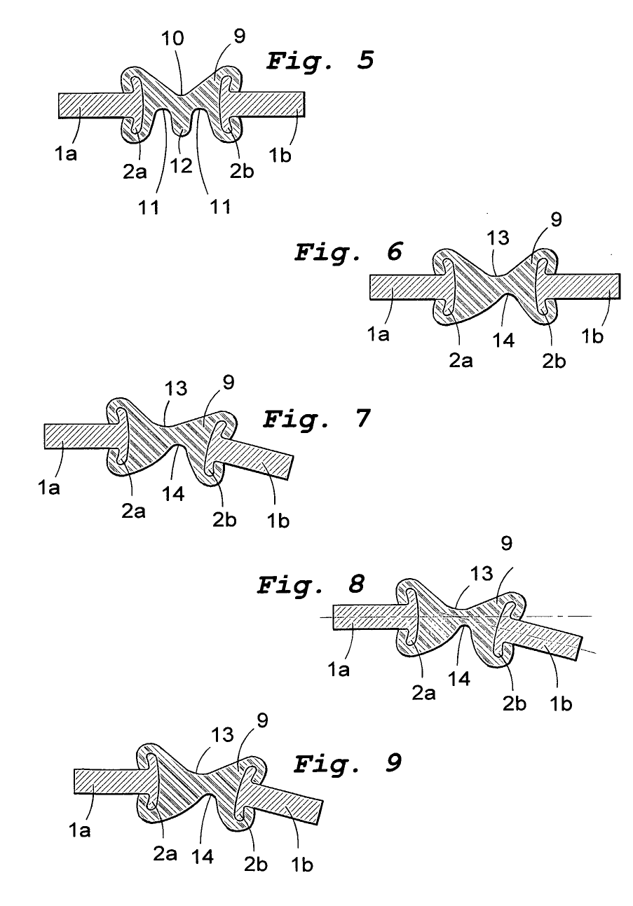

In FIGS. 5-9, some examples of embodiments of joint prostheses are shown in longitudinal section where the profile of the joint body in various ways has a profile that supports the possibility of achieving an angled neutral position of the joint prosthesis and that contributes to achieve a favourable characteristic from the neutral position as regards the relation between applied muscular force and angulation upon movement in different directions.

In the example according to FIG. 5, on one side the joint body 9 has one concavity 10 arranged in the centre and on the opposite side two concavities 11, spaced-apart by a protruding portion 12.

In the example according to FIG. 6, on one side the joint body 9 has a concavity 13 having a relatively large radius of curvature. On the other side there is a concavity 14 having a smaller radius of curvature and being displaced laterally in the longitudinal direction of the joint.

FIG. 7 shows an example similar to that of FIG. 6 with the anchoring axes being angled in the cross-sectional plane.

In FIGS. 8 and 9, additional examples are shown with the concavities 13, 14 having mutually different radii of curvature and different positions in relation to each other and in relation to the centre of the joint body 9 in the longitudinal direction.

In FIG. 10, an additional example of a finger joint prosthesis is shown in a side view with suitable dimensions of the profile of the joint body being indicated in mm.

FIG. 11 is an end view of the joint prosthesis in FIG. 10.

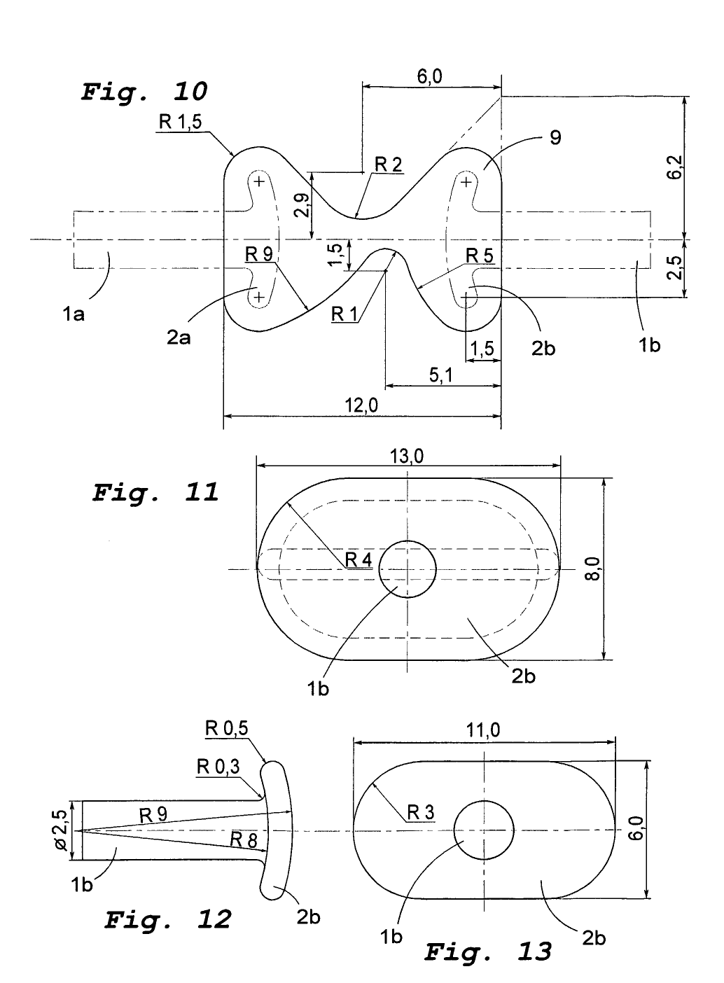

In the examples above, the end flanges 2 of the anchoring elements 1 are curved. In FIG. 12, a side view is shown more in detail and including indications of dimension when the same are adapted to a finger joint prosthesis. FIG. 13 is an end view of the anchoring element 1 in FIG. 12.

The material in the joint body 9 may suitably be an elastomer of the type denominated Chronoflex™ 80 shore A or Pellethane™ 85 shore A.

1. Joint prosthesis comprising a first anchoring element a second anchoring element and a joint body which joint body is of an elastic material and arranged so that the joint prosthesis is deflectable at least in a first plane of bending, and which joint body is attached to said first and second anchoring elements, each of which anchoring elements are arranged to be secured into a respective bone having a respective anchoring axis running in the longitudinal axis of the respective bone, which anchoring axes do not coincide with each other when the joint prosthesis is in an unloaded state characterized in that the joint body is arranged so that the joint prosthesis is deflectable also in a second plane of bending, perpendicular to said first plane of bending, the moment of resistance of the joint body for bending in the first plane of bending being smaller than the moment of resistance thereof in the second plane of bending.

2. Joint prosthesis according to claim 1, characterized in that said anchoring axes in a projection in said first plane of bending, form a first angle to each other.

3. Joint prosthesis according to claim 2, characterized in that said first angle is in the interval of 5-30°, preferably in the interval of 10-20°.

4. Joint prosthesis according to claim 1, characterized in that said anchoring axes in a projection in said second plane of bending, form a second angle to each other and that said first angle is larger than said second angle

5. Joint prosthesis according to claim 1, characterized in that said first and second anchoring axes are parallel to each other.

6. Joint prosthesis according to claim 1, characterized in that the joint body comprises a first bordering surface and a second bordering surface, located on one side each of the projection of the anchoring axes in said first plane of bending, which bordering surfaces in a cross section in said first plane of bending have different outlines, and that each outline has at least one part being concave up against said projection of the bending axes.

7. Joint prosthesis according to claim 6, characterized in that the concave part of the first bordering surface has another radius of curvature than the concave part of the second bordering surface.

8. Joint prosthesis according to claim 6, characterized in that the distances from the anchoring axis of the first anchoring element to the concave part of the respective bordering surface are differently large.

9. Joint prosthesis according to claim 6, characterized in that at least one of said concave parts is located more distant from the first anchoring element than the second anchoring element.

10. Joint prosthesis according to claim 1, characterized in that at least one of the anchoring elements comprises an end flange secured in the joint body which flange has a curved shape including a convex surface facing the central part of the joint body.

11. Joint prosthesis according to claim 1, characterized in that it is a finger joint prosthesis.

12. Use of a joint prosthesis according to claim 1 for replacing a defective joint of a person.

13. A method related to the application of a joint prosthesis of a kind including an elastic joint body attached to a first and a second anchoring element, whereby the anchoring elements are secured to a respective bone and having a respective anchoring axis running in the longitudinal axis of the respective bone, which anchoring axis do not coincide with each other when the joint prosthesis is in an unloaded state and arranging the joint body such that the joint prosthesis is deflectable at least in a first plane of bending characterized by arranging the joint body so that the joint prosthesis is deflectable also in a second plane of bending, perpendicular to said first plane, the moment of resistance of the joint body for bending the first plane of bending being smaller than the moment of resistance thereof in the second plane of bending.

14. Method according to claim 13, characterized in that it is exercised by the use of a joint prosthesis according to claim 1.
